(19) United States
(12) Patent Application Publication
(10) Pub. No.: US 2006/0199279 A1
(43) Pub. Date: Sep. 7, 2006

(54) Methods for identifying post-translationally modified polypeptides

(76) Inventors: Viorica Lopez-Avila, Loveland, CO (US); David L. Hirschberg, Loveland, CO (US); James A. Apffel JR., Loveland, CO (US)

Correspondence Address: AGILENT TECHNOLOGIES, INC.;INTELLECTUAL PROPERTY ADMINISTRATION, LEGAL DEPT., M/S DU404, P.O. BOX 7599, LOVELAND, CO, 80537-0599, US

(21) Appl. No.: 11/074,472

(22) Filed: Mar. 7, 2005

(51) Int. Cl. G01N 33/543; G01N 33/00

(52) U.S. Cl. 436/518; 436/86

(57) ABSTRACT

The invention provides methods of analyzing a sample. In general, the methods involve multi-dimensionally fractionating a sample to produce a set of sub-fractions, identifying a sub-fraction of interest by evaluating binding of a first portion of the sub-fractions to a binding agent; and analyzing the mass of analytes in a second portion of the sub-fraction of interest. Also provided is a system for performing the subject methods. The invention finds use in a variety of different medical, research and proteomics applications.

### BACKGROUND OF THE INVENTION

Post-translational modification of a protein in a cell involves the enzymatic addition of a chemical group, e.g., a phosphate or glycosyl group, to an amino acid of that protein. Such modifications are thought to be required for maintaining and regulating protein structure and function, and abnormal post-translational events have been detected in a wide variety of diseases and conditions, including heart disease, cancer, neurodegenerative and inflammatory diseases and diabetes.

Protein phosphorylation is a type of post-translational modification used to selectively transmit regulatory signals from receptors positioned at the surface of a cell to the nucleus of the cell. The molecules mediating these reactions are predominantly protein kinases that catalyze the addition of phosphate groups onto selected proteins, and protein phosphatases that catalyze the removal of those phosphate groups. Complex biological processes such as cell cycle, cell growth, cell differentiation, and metabolism are orchestrated and tightly controlled by reversible phosphorylation events that modulate protein activity, stability, interactions and localization. Accordingly, protein phosphorylation is thought to play a regulatory role in almost all aspects of cell biology. Perturbations in protein phosphorylation, e.g. by mutations that generate constitutively active or inactive protein kinases and phosphatases, play a prominent role in oncogenesis. Serine, threonine, tyrosine, histidine, arginine, lysine, cysteine, glutamic acid or aspartic acid residues may be phosphorylated. The hydroxyl groups of serine, threonine or tyrosine residues are most commonly phosphorylated.

Protein glycosylation, on the other hand, is acknowledged as being a post-translational modification that has a major effect on protein folding, conformation distribution, stability and activity. Carbohydrates in the form of asparagine-linked (N-linked) or serine/threonine (O-linked) oligosaccharides are major structural components of many cell surface and secreted proteins. All N-linked carbohydrates are linked through N-acetylglucosamine, and most O-linked carbohydrates are attached through N-acetylgalactosamine. O-linked N-acetylglucosamine (O-GlcNAc) is a recently identified type of glycosylation. Unlike classical O- or N-linked protein glycosylation, O-GlcNAc glycosylation involves linking a single GlcNAc moiety to the hydroxyl group of a serine or threonine residue. Increasing evidence suggests that O-GlcNAc modification is a regulatory modification similar to phosphorylation, since it is highly dynamic and rapidly cycles in response to cellular signals.

Because of the central role of post-translational modification in cell biology, much effort has been focused on the development of methods for identifying post-translationally modified proteins. A variety of methods for identifying and characterizing post-translationally modified proteins have been developed.

For example, traditional methods for analyzing phosphorylation sites involve incorporation of radioactive phosphorus into cellular phosphorylated proteins by feeding cells with ³²p ATP. The radioactive proteins can be detected during subsequent fractionation procedures (e.g. two-dimensional gel electrophoresis or high-performance liquid chromatography). Proteins thus identified can be subjected to complete hydrolysis and the phosphoamino acid content determined. The site(s) of phosphorylation can be determined by proteolytic digestion of the radiolabeled protein, separation and detection of phosphorylated peptides (e.g., by two-dimensional peptide mapping), followed by peptide sequencing by Edman degradation. These techniques are generally tedious, require significant quantities of the phosphorylated protein and involve the use of considerable amounts of radioactivity.

In recent years, affinity chromatography has become widely employed in many of methods for identifying post-translational modifications. The most widely used method involves selectively enriching phosphoproteins from a sample using immobilized metal affinity chromatography (IMAC). In this technique, metal ions, usually Fe³⁺ or Ga³⁺, are bound to a chelating support. Phosphoproteins are selectively bound to the column by the affinity of the phosphate moiety of the phosphoproteins to the metal ions of the column. The phosphoproteins can be released using high pH buffer, and subjected to mass spectrometry (MS) analysis. While this method is widely employed, it is limited because many phosphoproteins are unable to bind to IMAC columns, and bound phosphoproteins are often difficult to elute from such columns. Furthermore, these methods produce significant background signals from unphosphorylated proteins that are typically acidic in nature and therefore have affinity for the immobilized metal ions of such columns.

Accordingly, there is an ongoing need for straightforward and reliable methods to identify post-translationally modified proteins in a sample. This invention meets this need, and others.

Publications of interest include: Watts et al. (J. Biol. Chem 1994 269:29520); Schlosser et al. (Proteomics 2002 2:911-918); Oda et al. (Nature Biotechnol. 2001 19, 379-382), Zhou et al. (Nature Biotech. 2001 19: 375-378); Link (Trends in Biotechnology 2002 20:S8-S13); Yan et al. (Proteomics 2003 3:1228-35, Zhang et al (Anal Chem. 1998 70:2050-9), Cantin et al. (J. Chromatogr. A. 2004 1053:7-14) and WO0157530.

### SUMMARY OF THE INVENTION

The invention provides methods of analyzing a sample. In general, the methods involve multi-dimensionally fractionating a sample to produce a set of sub-fractions, identifying a sub-fraction of interest by evaluating binding of a first portion of the sub-fractions to a binding agent; and analyzing the mass of analytes in a second portion of the sub-fraction of interest. In certain embodiments, the methods involve depositing a first portion of the sub-fractions on a substrate to produce an array, and interrogating the array with a post-translational modification indicator. Also provided is a system for performing the subject methods. The invention finds use in a variety of different medical, research and proteomics applications.

### BRIEF DESCRIPTION OF THE FIGURES

FIG. 1 is a flow diagram describing one embodiment of the subject invention.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “sample” as used herein relates to a material or mixture of materials, typically, although not necessarily, in fluid form, e.g., aqueous, containing one or more components of interest. Samples may be derived from a variety of sources such as from food stuffs, environmental materials, a biological sample such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components).

Components in a sample are termed “analytes” herein. In certain embodiments, the sample is a complex sample containing at least about 10², 5×10², 10³, 5×10³, 10⁴, 5×10⁴, 10⁵, 5×10⁵, 10⁶, 5×10⁶, 10⁷, 5×10⁷, 10⁸, 10⁹, 10¹⁰, 10¹¹, 10¹² or more species of analyte.

The term “analyte” is used herein to refer to a known or unknown component of a sample, which will specifically bind to a capture agent on a substrate surface if the analyte and the capture agent are members of a specific binding pair. In general, analytes are biopolymers, i.e., an oligomer or polymer such as an oligonucleotide, a peptide, a polypeptide, an antibody, or the like. In this case, an “analyte” is referenced as a moiety in a mobile phase (e.g., fluid), to be detected by a “capture agent” which, in some embodiments, is bound to a substrate, or in other embodiments, is in solution. However, either of the “analyte” or “capture agent” may be the one which is to be evaluated by the other (thus, either one could be an unknown mixture of analytes, e.g., polypeptides, to be evaluated by binding with the other).

A “biopolymer” is a polymer of one or more types of repeating units, regardless of the source. Biopolymers may be found in biological systems and particularly include polypeptides and polynucleotides, as well as such compounds containing amino acids, nucleotides, or analogs thereof. The term “polynucleotide” refers to a polymer of nucleotides, or analogs thereof, of any length, including oligonucleotides that range from 10-100 nucleotides in length and polynucleotides of greater than 100 nucleotides in length. The term “polypeptide” refers to a polymer of amino acids of any length, including peptides that range from 6-50 amino acids in length and polypeptides that are greater than about 50 amino acids in length.

In most embodiments, the terms “polypeptide” and “protein” are used interchangeably. The term “polypeptide” includes polypeptides in which the conventional backbone has been replaced with non-naturally occurring or synthetic backbones, and peptides in which one or more of the conventional amino acids have been replaced with one or more non-naturally occurring or synthetic amino acids. The term “fusion protein” or grammatical equivalents thereof references a protein composed of a plurality of polypeptide components, that while not attached in their native state, are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, P-galactosidase, luciferase, and the like.

In general, polypeptides may be of any length, e.g., greater than 2 amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally greater than 2 amino acids, greater than 4 amino acids, greater than about 10 amino acids, greater than about 20 amino acids, usually up to about 50 amino acids. In some embodiments, peptides are between 5 and 30 amino acids in length.

The term “capture agent” refers to an agent that binds an analyte through an interaction that is sufficient to permit the agent to bind and concentrate the analyte from a homogeneous mixture of different analytes. The binding interaction may be mediated by an affinity region of the capture agent. Representative capture agents include polypeptides and polynucleotides, for example antibodies, peptides or fragments of single stranded or double stranded DNA may employed. Capture agents usually “specifically bind” one or more analytes.

Accordingly, the term “capture agent” refers to a molecule or a multi-molecular complex which can specifically bind an analyte, e.g., specifically bind an analyte for the capture agent, with a dissociation constant (K_{D}) of less than about 10⁻⁶ M without binding to other targets.

The term “specific binding” refers to the ability of a capture agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold). In certain embodiments, the affinity between a capture agent and analyte when they are specifically bound in a capture agent/analyte complex is characterized by a K_{D} (dissociation constant) of less than 10⁻⁶ M, less than 10⁻⁷ M, less than 10⁻⁸ M, less than 10⁻⁹ M, usually less than about 10⁻¹⁰ M.

The term “capture agent/analyte complex” is a complex that results from the specific binding of a capture agent with an analyte, i.e., a “binding partner pair”. A capture agent and an analyte for the capture agent specifically bind to each other under “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens, are well known in the art (see, e.g., Harlow and Lane (Antibodies: A Laboratory Manual Cold Spring Harbor Laboratory, Cold Spring Harbor, N.Y. (1989)). Conditions suitable for specific binding typically permit capture agents and target pairs that have a dissociation constant (K_{D}) of less than about 10⁻⁶ M to bind to each other, but not with other capture agents or targets.

As used herein, “binding partners” and equivalents refer to pairs of molecules that can be found in a capture agent/analyte complex, i.e., exhibit specific binding with each other.

The phrase “surface-bound capture agent” refers to a capture agent that is immobilized on a surface of a solid substrate, where the substrate can have a variety of configurations, e.g., a sheet, bead, or other structure, such as a plate with wells. In certain embodiments, the collections of capture agents employed herein are present on a surface of the same support, e.g., in the form of an array.

The term “pre-determined” refers to an element whose identity is known prior to its use. For example, a “pre-determined analyte” is an analyte whose identity is known prior to any binding to a capture agent. An element may be known by name, sequence, molecular weight, its function, or any other attribute or identifier. In some embodiments, the term “analyte of interest”, i.e., an known analyte that is of interest, is used synonymously with the term “pre-determined analyte”.

The terms “antibody” and “immunoglobulin” are used interchangeably herein to refer to a capture agent that has at least an epitope binding domain of an antibody. These terms are well understood by those in the field, and refer to a protein containing one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)). Monoclonal antibodies and “phage display” antibodies are well known in the art and encompassed by the term “antibodies”.

The term “mixture”, as used herein, refers to a combination of elements, e.g., capture agents or analytes, that are interspersed and not in any particular order. A mixture is homogeneous and not spatially separable into its different constituents. Examples of mixtures of elements include a number of different elements that are dissolved in the same aqueous solution, or a number of different elements attached to a solid support at random or in no particular order in which the different elements are not specially distinct. In other words, a mixture is not addressable. To be specific, an array of capture agents, as is commonly known in the art and described below, is not a mixture of capture agents because the species of capture agents are spatially distinct and the array is addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

The term “array” encompasses the term “microarray” and refers to an ordered array of capture agents for binding to aqueous analytes and the like.

An “array,” includes any two-dimensional or substantially two-dimensional (as well as a three-dimensional) arrangement of spatially addressable regions (i.e., “features”) containing capture agents, particularly antibodies, and the like. Where the arrays are arrays of proteinaceous capture agents, the capture agents may be adsorbed, physisorbed, chemisorbed, or covalently attached to the arrays at any point or points along the amino acid chain. In some embodiments, the capture agents are not bound to the array, but are present in a solution that is deposited into or on features of the array.

Any given substrate may carry one, two, four or more arrays disposed on a surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of the same or different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations. The term “array” encompasses the term “microarray” and refers to any one-dimensional, two-dimensional or substantially two-dimensional (as well as a three-dimensional) arrangement of spatially addressable regions, usually bearing biopolymeric capture agents, e.g., polypeptides, nucleic acids, and the like.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm.

Arrays can be fabricated using drop deposition from pulse-jets of either precursor units (such as nucleotide or amino acid monomers) in the case of in situ fabrication, or the previously obtained capture agent.

An array is “addressable” when it has multiple regions of different moieties (e.g., different capture agent) such that a region (i.e., a “feature” or “spot” of the array) at a particular predetermined location (i.e., an “address”) on the array will detect a particular sequence. Array features are typically, but need not be, separated by intervening spaces.

An “array layout” refers to one or more characteristics of the features, such as feature positioning on the substrate, one or more feature dimensions, and an indication of a moiety at a given location.

The term “fractionate” refers to the separation of a liquid composition into distinct, different liquid fractions via chromatography. The “fractions” of a fractionated sample each contain a different set of analytes, although certain analytes may be present in more than one fraction of the fractionated sample.

The term “multi-dimensionally fractionated sample” refers to a sample that has been fractioned by at least two different chromatography methods. In one exemplary embodiment provided to illustrate what is meant by this term, a “multi-dimensionally fractionated sample” is a sample that has been fractionated by ion exchange chromatography (i.e., fractionated in a first dimension) and by reverse phase chromatography (i.e., fractionated in a second dimension). In this example, the fractions produced by ion exchange chromatography are fractionated by reverse phase chromatography to produce sub-fractions. Methodologies for making multi-dimensionally fractionated samples are well known in the art (see, e.g., Apffel, A. “Multidimensional Chromatography of Intact Proteins” in Purifying Proteins for Proteomics: A Laboratory Manual, Richard Simpson (ed.), Cold Spring Harbor Press, 2003).

The term “sub-fraction” refers to a type of fraction obtained after a sample has been multi-dimensionally fractionated (i.e., fractionated by at least two different chromatography devices). A “sub-fraction” is therefore a fraction obtained by fractionation of a fraction, using a second chromatography device.

[042] A “portion” of a liquid composition is part of a liquid composition. A portion of a liquid composition may be removed from the liquid composition (e.g., by pipetting from the composition), or portions of a liquid composition may be made by dividing the liquid composition. All of the portions of a composition generally contain the same molecules at the same relative concentrations (excluding any molecules that may have evaporated of may have been changed or removed during processing of the composition).

The term “post-translational modification indicator”, as will be described in greater detail below, is any molecule that can indicate the presence of a post-translational modification on an analyte.

A “post-translationally modified sub-fraction” is a sub-fraction containing a post-translationally modified analyte.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

### DETAILED DESCRIPTION OF THE INVENTION

The invention provides methods of analyzing a sample. In general, the methods involve multi-dimensionally fractionating a sample to produce a set of sub-fractions, identifying a sub-fraction of interest by evaluating binding of a first portion of the sub-fractions to a binding agent; and analyzing the mass of analytes in a second portion of the sub-fraction of interest. In certain embodiments, the methods involve depositing a first portion of the sub-fractions on a substrate to produce an array and interrogating the array with a post-translational modification indicator. Also provided is a system for performing the subject methods. The invention finds use in a variety of different medical, research and proteomics applications.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Reference to a singular item includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In further describing the subject invention, the subject methods are described first, followed by a description of a system for analyzing a sample in which the subject methods find use.

Methods for Sample Analysis

The invention provides a method for sample analysis. In general terms, the subject method involves fractionating a sample in at least two dimensions (i.e., using at least two different chromatography devices) to produce a set of sub-fractions, identifying a sub-fraction of interest (i.e., a sub-fraction containing an analyte of known or unknown identity that is to be further investigated) by evaluating a binding activity of a portion of each sub-fraction of the set of sub-fractions, and analyzing the masses of analytes in a second portion of the sub-fraction of interest.

The subject methods may be performed in a variety of different ways. For example, in certain embodiments, a sub-fraction containing a pre-determined analyte of interest (e.g., a particular polypeptide) is first identified by its binding to a capture agent specific for that analyte. The identified sub-fraction is subjected to mass analysis to assess post-translational modification of that analyte. For example, in certain embodiments, a portion of each sub-fraction from a set of sub-fractions may be deposited onto a substrate to produce an array, and the array contacted with a binding agent, e.g., an antibody that specifically binds to a pre-determined analyte of interest. Binding of the binding agent to a sub-fraction of interest identifies the sub-fraction of interest. Post-translational modification of an analyte of interest may be assessed by analyzing data obtained subjecting a second portion of that sub-fraction to mass analysis to assess. For example, mass spectrometry may be employed to assess post-translational modification of the analyte of interest (including determining whether or not the analyte is post-translationally modified or determining the amount of post-translationally modified analyte). In an alternative embodiment, the sub-fraction containing a pre-determined analyte may be identified by labeling each of the sub-fractions and contacting the labeled sub-fractions with an array of analyte-specific capture agents (e.g., an array of antibodies that bind to specific analytes).

In other embodiments that will be described in greater detail below, a set of sub-fractions are deposited onto a substrate to form an array. The array is interrogated with a binding agent to identify a post-translationally modified sub-fraction of interest (e.g., a sub-fraction containing a post-translationally modified analyte of unknown identity), and that sub-fraction is subjected to mass analysis to identify (e.g., determine the identity of) the post-translationally modified analyte of interest. The mass analysis may also provide an evaluation of the amount of post-translationally modified analyte in the sub-fraction.

In one embodiment, every sub-fraction produced by a multi-dimensional fractionation system is subjected to mass analysis to produce data, and the mass data for only sub-fractions of interest is assessed. In other embodiments, only sub-fractions of interest are subjected to mass analysis.

In certain embodiments, this method involves producing an array of sub-fractions and interrogating the array with a binding agent, e.g., a labeled binding agent, such as a polypeptide binding agent, e.g., a labeled antibody or peptide, or an indicator, e.g., a post-translational modification indicator. In one embodiment, a sub-fraction of interest is ionized and subjected to mass spectrometry in order to analyze the masses of analytes in that sub-fraction.

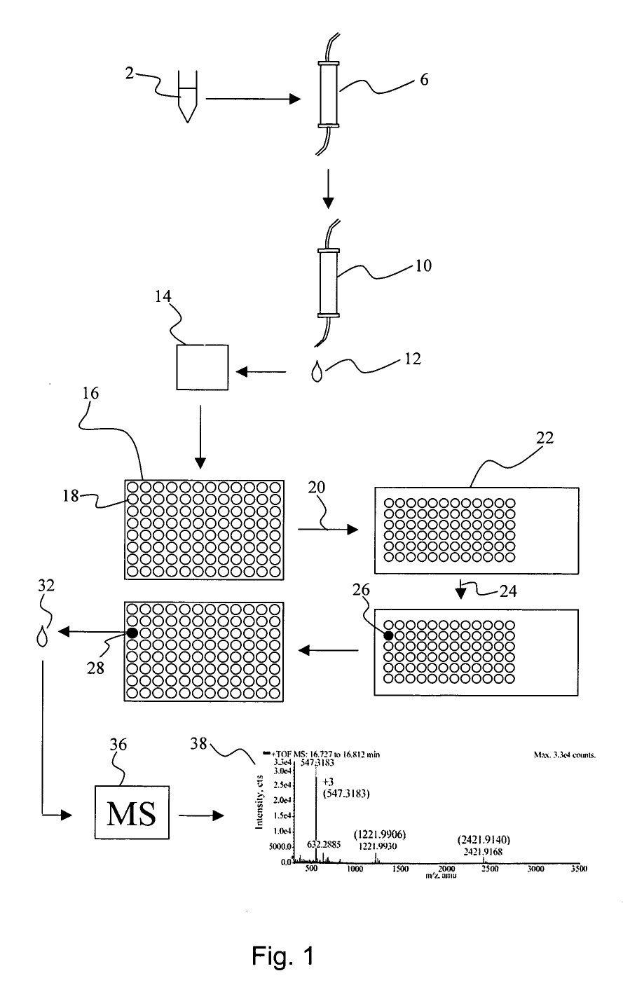

With reference to FIG. 1, showing an exemplary embodiment not intended to limit the invention, the method may involve producing a multi-dimensionally fractionated sample by fractionating a sample 2 using a first chromatography device 6 to produce a plurality of fractions, and fractionating those fractions using a second chromatography device 10 to produce a set of sub-fractions 12. The sub-fractions are individually placed into the vessels 18 of an addressable storage system 16 (e.g., the wells of a multi-well plate or the like), typically using a fraction collector 14. Portions of the sub-fractions are then deposited 20 onto a surface of a substrate and linked thereto to produce an addressable array 22 of sub-fractions. The array is then contacted with a binding agent 24, e.g., a post-translational modification (PTM) indicator to identify a feature containing a sub-fraction of interest 26, e.g., a sub-fraction containing a post-translationally modified polypeptide. The vessel of the addressable storage system containing the sub-fraction of interest 28 is identified, and a portion 32 of that sub-fraction is subjected to mass analysis, e.g., using mass spectrometry 36 to produce data 38 regarding the identity of an analyte in the sub-fraction, e.g., a post-translationally modified polypeptide. The identity of the analyte bound by the binding agent can be determined using this data.

In describing these methods in greater detail, the multi-dimensional fractionation methods will be described first, followed by a discussion of how arrays may be fabricated using sub-fractions produced by the multi-dimensional fractionation methods. Finally, methods of identifying sub-fractions of interest, e.g., sub-fractions containing post-translationally modified polypeptides, will be described.

Multi-Dimensional Fractionation

The subject methods of sample analysis involve multi-dimensional fractionation of a sample. In general, multi-dimensional fractionation methods employ at least two different liquid chromatography devices (termed herein as a “first” chromatography device and “second” chromatography device), and the sample is fractionated using both of those devices. A sample is fractionated by a first chromatography device to produce fractions, and those fractions are themselves fractionated by a second chromatography device to produce sub-fractions. The sub-fractions produced by the second chromatography device are then used in the remainder of the methods, as will be discussed in greater detail below.

For many purposes, any two or more different liquid chromatography devices may be used to multi-dimensionally fractionate a sample. Accordingly, there are many liquid chromatography devices that may be employed in the subject methods including, but not limited to: a) hydrophobic interaction chromatography devices (e.g., normal or reverse phase chromatography devices that employ a hydrophobic column, for example a C4, C8 or C18 column), b) ion exchange chromatography devices (e.g., anion exchange or cation exchange (including strong cation exchange) devices that employ, for example, a diethyl aminoethyl (DEAE) or carboxymethyl (CM) column), c) affinity chromatography devices (e.g., any chromatography device having a column linked to a specific binding agent such as a polypeptide, a nucleic acid, a polysaccharide or any other molecule such as, for example a chelated metal (e.g., chelated Fe³⁺ or Ga³⁺) and IMAC columms), and d) gel filtration chromatography devices (e.g., any chromatography device containing a size excluding gel such as SEPHADEX™ or SEPHAROSE™ of any pore size) that separate analytes in a sample on the basis of their size. High performance liquid chromatography (HPLC) or capillary devices are employed in certain embodiments of the invention.

The particular chromatography conditions employed with any of the above types of chromatography devices (e.g., the binding, wash or elution buffers used, the salt or solvent gradients used, whether step or continuous gradient is used, the exact column used, and the run-time etc.), are well known in the literature and are readily adapted to the instant methods without undue effort.

The first and second chromatography devices employed in the subject methods are generally “different” to each other in that they use different physical properties to separate the analytes of a sample. Analyte size, analyte affinity to a substrate, analyte hydrophobicity and analyte charge are exemplary properties that are different to each other. Accordingly, a sample may be first fractionated using a device selected from a hydrophobic interaction chromatography device, an ion exchange chromatography device, an affinity chromatography device or a gel filtration chromatography device to produce fractions, and the resultant fractions are then themselves fractionated by a different device. In one exemplary embodiment, a sample is first subjected to ion exchange chromatography to produce fractions, and those fractions are subject to reverse phase chromatography to produce sub-fractions.

The number of fractions produced by each of the chromatography devices employed may vary depending on the complexity of the sample to be analyzed and the particular fractionation devices used. In certain embodiments, the first chromatography device produces at least 5 (e.g., at least 10, at least 50, at least 100, at least 200, at least 500, usually up to about 500 or 1,000 or more) fractions, and each of those fractions is further fractionated into at least 5 (e.g., at least 10, at least 50, at least 100, at least 200, at least 500, usually up to about 500 or 1,000 or more) sub-fractions by the second chromatography device. In general, a sample may be multi-dimensionally fractionated into any number of sub-fractions (e.g., at least 100, at least 500, at least 1,000, at least 5,000 or at least 10,000 usually up to about 50,000 or 100,000 fractions or more). In certain embodiments, the sub-fractions of a sample may contain, on average, less than about 10 (e.g., about 1, 2, 4, 6 or 8) different polypeptides.

In general, multi-dimensional fractionation systems readily adaptable for employment in the instant methods are known in the art. Further details of these multi-dimensional fractionation methods may be found in Wang et al. (Mass Spectrom Rev. 2004 June 30; Epub ahead of print); Wang et al. (J. Chromatogr. 2003 787:11-8); Issaq et al. (Electrophoresis 2001 22:3629-38); Wolters et al. (Anal Chem. 2001 73:5683-90); and Link (Trends in Biotechnology 2002 20:S8-S13), for example.

As is known in the art, the output of a first chromatography device of a multi-dimensional chromatography system may be linked to the input of the second chromatography device of the system. In such a system, the fractions produced by the first device are further fractionated by the second device immediately after they are input into the second device from the first device. Accordingly, multi-dimensional fractionation of a sample may be continuous in that the devices employed are operating at the same time.

The sub-fractions of a sample may be individually deposited into the addressable storage system using a fraction collector. In certain embodiments, the collected sub-fractions may be concentrated and/or stored prior to use.

Identification of a Sub-Fraction of Interest

A portion of each of the sub-fractions (i.e., a part of each of the sub-fractions) produced by the above multi-dimensional fractionation methods is tested for its ability to bind to a binding agent. This portion is generally referred to herein as a “first portion” to distinguish it from another portion (e.g., a “second portion”) of the sub-fraction that may be used in mass analysis of the sub-fraction. The use of the terms “first” and “second” are not used to indicate any sequence of events in a method (e.g., that the first portion is assessed prior to a second portion being assessed). In fact, the first and second portions of a sample may be assessed in any order (e.g., the first or the second portion may be assessed prior to assessment of the other portion).

In one embodiment, a first portion of each of the sub-fractions is deposited onto the surface of a substrate to produce an array, and this array is contacted with the binding agent. Each sub-fraction is represented by a different feature, and the features of a subject array contain the polypeptides of each sub-fraction deposited thereon. A subject array generally comprises a plurality (e.g., at least 100, at least 500, at least 1000, at least 5000, usually up to about 10,000 or 50,000 or more) of spatially addressable features each containing one or more polypeptides of a sub-fraction. In certain embodiments therefore, a single species of polypeptide may be present in each of the features of a subject array. However, depending on the precise multi-dimensional fractionation method employed, a feature may contain a mixture of different polypeptides.

Methods for making arrays of polypeptides using contact and inkjet (i.e., piezoelectric) deposition methods are generally well known in the art (see e.g., U.S. Pat. Nos. 6,372,483, 6,352,842, 6,346,416 and 6,242,266; MacBeath and Schreiber, Science (2000) 289:1760-3) and do not need to be described here in any more detail.

Once an array of sub-fractions has been fabricated, the array is contacted with a binding agent, e.g., a labeled antibody or polypeptide or the like to identify a feature containing an analyte of interest. The binding agent is generally a pre-determined binding agent, i.e., an agent whose identity is known prior to use. In one embodiment the array is contacted with a post-translational modification indicator to identify a feature containing a post-translationally modified polypeptide. Once such a feature is identified, a second portion of the sub-fraction deposited at that feature is subjected to mass analysis, e.g., mass spectrometry analysis, to produce data. The data may be analyzed to identify the analyte of interest.

A variety of binding agents may be employed in the subject methods. In particular embodiments, a post-translational modification indicator, e.g., a labeled antibody or post-translational modification-specific dye may be used. For example, to identify phosphoproteins (i.e., polypeptides to which a phosphate group has been added), any one or more of a variety of labeled anti-phosphotyrosine, anti-phosphoserine or anti-phosphothreonine antibodies may be used. Such antibodies may be purchased from a variety of different manufacturers, including Research Diagnostics Inc. (Flanders N.J.), Zymed Laboratories, Inc. (San Francisco, Calif.), PerkinElmer (Torrance, Calif.) and Sigma-Aldrich (St. Louis, Mo.). Alternatively, dyes (particularly fluorescent dyes) that specifically bind to phosphoproteins may be employed. Such dyes include methyl green (Cutting et al, Analytical Biochemistry 1973 54, 386-394) sold by Pierce (Rockford, Ill.), among others, and the phosphopeptide-specific PRO-Q DIAMOND™ dye of Molecular Probes of Invitrogen Corp. (Eugene, Oreg.). Likewise, to identify glycoproteins, one or more of a variety of anti-glycoprotein antibodies may be employed (see product literature for Novus (Littleton, Colo.) and Sigma-Aldrich (St. Louis, Mo.), for example). A variety of glyco-specific dyes, e.g., SYPRO™ Ruby and PRO-Q EMERALD™ dyes of Molecular Probes of Invitrogen Corp. (Eugene, Oreg.) may also be employed.

The methods generally involve contacting a subject array with a binding agent under conditions suitable for specific binding of the analytes deposited onto the array. The array is read using an array reader (e.g., an array scanner), and features that contain an analyte of interest are identified. Details of scanners and scanning procedures that may be employed in the subject methods are found in U.S. Pat. Nos. 6,806,460, 6,791,690 and 6,770,892, for example.

Once a feature containing an analyte of interest is identified, the address of that feature may be determined (usually by reference to column and row coordinates, as well as an array number if more than one array is present on the substrate). The address of that feature is used to identify the address of the vessel of the addressable storage system containing the sub-fraction deposited to that feature. The address of the vessel of the addressable storage system may be identified by a variety of means, including by using a look-up table or the like. Once the vessel containing a sub-fraction of interest identified, a second portion of the sub-fraction of interest is subjected to molecular mass analysis.

In particular embodiments, a portion (e.g., 100 nl, 500 nl, 1 μl, 2 μl, 5 μl, usually up to 10 μl or 100 μl or more) of the sub-fraction of interest is removed from the identified vessel, the analytes of the removed portion are ionized and the resultant ions are investigated by mass spectrometry.

In particular embodiments, the analytes of a sub-fraction of interest are analyzed using any mass spectrometer that has the capability of measuring analyte, e.g., polypeptide, masses with high mass accuracy, precision, and resolution. Accordingly, the isolated analytes may be analyzed by any one of a number of mass spectrometry methods, including, but not limited to, matrix-assisted laser desorption ionization time-of-flight mass spectrometry (MALDI-TOF), triple quadrupole MS using either electrospray MS, electrospray tandem MS, nano-electrospray MS, or nano-electrospray tandem MS, as well as ion trap, Fourier transform mass spectrometry, or mass spectrometers comprised of components from any one of the above mentioned types (e.g. quadrupole-TOF). For example, isolated analytes may be analyzed using an ion trap or triple quadrupole mass spectrometer. In many embodiments, MALDI-TOF instrument are used because they yield high accuracy peptide mass spectrum. If MALDI methods are used, the portion to be ionized is usually concentrated on the MALDI sample plate using standard technology, e.g., repeated sample spotting followed by evaporation, to a suitable concentration, e.g., 1-10 pMole/μL. In other embodiments, a liquid sample is ionized using an electrospray system. In certain cases it may be desirable to identify a particular analyte in a sub-fraction, in which case techniques such as selective ion monitoring (SIM) may be employed.

The output from the above analysis contains data relating to the mass, i.e., the molecular weight, of analytes in the sub-fraction of interest, and their relative or absolute abundances in the sample.

The analyte masses obtained from mass spectrometry analysis may analyzed to provide the identity of the analyte. In one embodiment, the obtained masses are compared to a database of molecular mass information to identify the analyte. In general, methods of comparing data produced by mass spectrometry to databases of molecular mass information to facilitate data analysis is very well known in the art (see, e.g., Yates et al, Anal Biochem. 1993 214:397-408; Mann et al, Biol Mass Spectrom. 1993 22:338-45; Jensen et al, Anal Chem. 1997 D69:4741-50; and Cottrell et al., Pept Res. 1994 7:115-24) and, as such, need not be described here in any further detail.

Accordingly, the identity of an analyte in a sub-fraction of interest may be obtained using mass spectrometry. Further details of exemplary mass spectrometry systems that may be employed in the subject methods may be found in U.S. Pat. Nos. 6,812,459, 6,723,98, 6,294,779 and RE36,892.

As is well known in the art, for each analyte, information obtained using mass spectrometry may be qualitative (e.g., showing the presence or absence of an analyte, or whether the analyte is present at a greater or lower amount than a control analyte or other standard) or quantitative (e.g., providing a numeral or fraction that may be absolute or relative to a control analyte or other standard). Accordingly, the relative levels of a particular analyte in two or more different sub-fractions may be compared.

In certain embodiments, at any stage of the methods set forth above, the analytes may be cleaved into analyte fragments prior to mass analysis. For example, the analytes of an identified sub-fraction of interest may be cleaved prior to mass analysis to provide sequence information. In certain embodiments, cleaved and uncleaved portions of a sub-fraction of interest may be separately assessed by mass analysis to determine the identity of an analyte therein. Fragmentation of analytes can be achieved by chemical means, e.g. using cyanogen bromide or the like, enzymatic means, e.g., using a protease enzyme such as trypsin, chymotrypsin, papain, gluc-C, endo lys-C, proteinase K, carboxypeptidase, calpain, subtilisin or pepsin or the like, or physical means, e.g., sonication or shearing. The cleavage agent can be immobilized in or on a support, or can be free in solution.

Likewise, at any point in the above-recited methods, a portion of an identified sub-fraction may be treated with a kinase (e.g., a specific or non-specific serine, threonine or tyrosine kinase) or a phosphatase (e.g., a specific or non-specific phospho-serine, phospho-threonine or phospho-tyrosine phosphatase such as an alkaline phosphatase) to verify that a particular phosphoprotein is present or absent in a subfraction. For example, an array (e.g., a duplicate of an array contacted with a phosphoprotein binding agent) may be treated with a kinase or phosphatase to add (in the case of arrays treated with a kinase) or remove (in the case of arrays treated with a phosphates) phosphate groups from polypeptides of the array. The presence of a particular phosphoproteins at a particular element of the array can be verified by comparing results obtained from binding a phosphoprotein binding agent to a treated array and to results obtained from binding a phosphoprotein binding agent to an untreated array. Likewise, prior to mass analysis, a portion of a sub-fraction identified as containing a phosphoprotein can be treated with a kinase or phosphatase to verify that the sub-fraction does, indeed, contain a phosphoprotein. In certain embodiments, a portion of a sub-fraction or an array may be first treated with a phosphatase, and then treated with a kinase to verify the presence of a phosphoprotein. Such methods are readily adapted from those methods already known in the art, such as those of Zhang et al (Anal Chem. 1998 70:2050-9).

Further, in certain embodiments, the sub-fractions of a sample may be stored (e.g., placed in a refrigerator or freezer) at any stage of the above methods.

System for Sample Analysis

The invention further provides a system for sample analysis. In general, the subject system contains: a) a multi-dimensional sample fractionation system for producing sub-fractions of a sample, b) a system for assessing binding of the sub-fractions to a binding agent, and c) a system for assessing analyte mass. In certain embodiments, a subject multi-dimensional sample fractionation system may contain an ion exchange chromatography device and reverse phase chromatography device that may be linked to each other, and, in particular embodiments, a fraction collector for depositing sub-fractions into a multi-vessel storage system (e.g., multi-well plates or the like). The system for assessing binding may contain a device for depositing material on an substrate to form an array (i.e., an “arrayer”) and an array reader. Particular binding agents, e.g., post-translational modification indicators may be employed in a subject system. The system for assessing analyte mass may be a mass spectrometer system containing an ion source, a mass spectrometer (e.g., a TOF spectrometer or an ion trap), and any necessary ion transport and detection devices present therein.

In certain embodiments of the invention, the multi-dimensional sample fractionation system produces sub-fractions of a sample that are deposited into a multi-vessel storage system using a fraction collector. A first portion of each of the sub-fractions is deposited onto the surface of a suitable substrate using the arrayer, and, after it has been contacted with the binding agent, the array is read in the array reader. After identifying a sub-portion of interest, a second portion of that sub-portion is subjected to mass analysis by a mass spectrometer.

The above system and methods may be performed by hand, i.e., manually. However, in certain embodiments, the subject methods may be performed using an automated system. An exemplary automated system for analyzing a sample contains the above-recited components, as well as a robot for transferring multi-vessel storage units from one place to another, and pipetting robots. Suitable pipetting robots include the following systems: GENESIS™ or FREEDOM™ of Tecan (Switzerland), MICROLAB 4000™ of Hamilton (Reno, Nev.), QIAGEN 8000™ of Qiagen (Valencia, Calif.), the BIOMEK 2000™ of Beckman Coulter (Fullerton, Calif.) and the HYDRA™ of Robbins Scientific (Hudson, N.H.).

Utility

The subject methods may be employed in a variety of diagnostic, drug discovery, and research applications that include, but are not limited to, diagnosis or monitoring of a disease or condition (where the analyte that binds to the binding agent is a marker for the disease or condition), discovery of drug targets (where the amount of an analyte that binds to the binding agent is modulated in a disease or condition and may be targeted for drug therapy), drug screening (where the effects of a drug are monitored by assessing the levels of the analyte that binds to the binding agent), determining drug susceptibility (where drug susceptibility is associated with a particular profile of binding analytes), discovery of new binding partners (where an analyte that binds to a binding agent has not been previously identified) and basic research (where is it desirable to identify the presence of a particular analyte in a sample, or, in certain embodiments, the relative levels of an analyte in two or more samples).

In particular embodiments, the instant methods may be used to identify post-translationally modified polypeptides, including polypeptides that have been phosphorylated or glycosylated. In these embodiments, a sample is analyzed using the above methods, and the identity of some or all of the post-translationally modified polypeptides in the sample can be determined. In certain embodiments, the subject methods may be employed to produce a “profile”, i.e., a series of data points on the amounts and/or identities, of post-translationally modified polypeptides for a sample.

In certain embodiments, a sample may be analyzed to determine if a particular post-translationally modified polypeptide is present in the sample.

In other embodiments, the post-translational modification profiles of two or more different samples may be compared to identify post-translational modification events that are associated with a particular disease or condition (e.g., a phosphorylation or glycosylation event that is induced by the disease or condition and therefore may be part of a signal transduction pathway implicated in that disease or condition). In other words, post-translational modification profiles of two or more different samples may be obtained using the above methods, and compared.

The different samples may consist of an “experimental” sample, i.e., a sample of interest, and a “control” sample to which the experimental sample may be compared. In many embodiments, the different samples are pairs of cell types or fractions thereof, one cell type being a cell type of interest, e.g., abnormal cells, and the other a control, e.g., normal, cell type. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g. before and after a stimulus on the cells) may be employed. In another embodiment of the invention, the experimental material is cells susceptible to infection by a pathogen such as a virus, e.g. human immunodeficiency virus (HIV), etc., and the control material is cells resistant to infection by the pathogen. In another embodiment of the invention, the sample pair is represented by undifferentiated cells, e.g., stem cells, and differentiated cells. The subject methods are particularly employable in methods of detecting phosphorylated serum proteins.

Accordingly, among other things, the instant methods may be used to link certain post-translational modifications (i.e., a certain modification of a certain protein) to certain physiological events.

In particular embodiments, the subject methods may be used to establish cellular signaling pathways that are employed transmit signals in a cell (e.g., from the exterior or interior of the cell to a cell nucleus, or from one protein in a cell to another, directly or indirectly). For example, the subject methods may be employed to determine the phosphorylation status of a protein in a cell (e.g., determine how much of a particular protein is phosphorylated at any moment in time), thereby indicating the activity of the kinase or phosphatase for which that protein is a substrate, even if the identity of the kinase or phosphatase is unknown. The substrates for a particular kinase or phosphatase may be identified by virtue of the fact that they should be phosphorylated or dephosphorylated by the same stimulus, at the same point in time. A signal transduction pathway for a particular stimulus may be determined by identifying all of the phosphorylation/dephosphorylation events for a particular stimulus, and determining when those events occur. Certain post-translational modifications that occur before other post-translational modifications (e.g., immediately after a stimulus) are generally upstream in a signal transduction pathway, whereas other post-translational modifications that occur after other post-translational modifications (e.g., long after a stimulus) are generally at the end of a signal transduction pathway.

Kits

Also provided by the subject invention are kits for practicing the subject methods, as described above. The subject kits contain at least a binding agent for evaluating binding of a first portion of a sub-fractions, e.g., a post-translational modification indicator, and reagent for analyzing analyte mass, e.g. a solvent, an analyte cleavage agent, or molecular mass standards or the like. The kit may also contain a database, which may be a table, on paper or in electronic media, containing molecular mass information for the analytes. In some embodiments, the kits contain programming to allow a robotic system to perform the subject methods, e.g., programming for instructing the automatic system discussed above. The various components of the kit may be present in separate containers or certain compatible components may be precombined into a single container, as desired.

In addition to above-mentioned components, the subject kits may further include instructions for using the components of the kit to practice the subject methods, i.e., to instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of sample analysis, comprising:
multi-dimensionally fractionating a sample to produce a set of sub-fractions;
identifying a sub-fraction of interest by evaluating binding of a first portion of said sub-fractions to a binding agent; and
analyzing the mass of analytes in a second portion of said sub-fraction of interest.

2. The method of claim 1, wherein said identifying sub-fraction of interest includes:
producing an array of said sub-fractions; and
interrogating said array with a binding agent.

3. The method of claim 1, wherein said binding agent is a labeled binding agent.

4. The method of claim 3, wherein said labeled binding agent is a post-translational modification indicator.

5. The method of claim 1, wherein said analyzing the mass of analytes includes subjecting said second portion of said sub-fraction of interest to mass spectrometry analysis.

6. The method of claim 1, wherein said analyzing the mass of analytes provides the identity of an analyte in said sub-fraction of interest.

7. A method of sample analysis, comprising:
interrogating an array of sub-fractions of a multi-dimensionally fractionated sample with a post-translational modification indicator; and
assessing any post-translationally modified sub-fractions by mass spectrometry.

8. The method of claim 7, wherein said method includes:
separating said sub-fractions of said multi-dimensionally fractionated sample into first portions and second portions,
depositing said first portions upon a substrate to make said array; and
accessibly storing said second portions.

9. The method of claim 8, wherein said assessing includes:
accessing a stored second portion of a post-translationally modified sub-fraction; and
obtaining a molecular mass measurement of an analyte in said second portion by mass spectrometry.

10. The method of claim 7, wherein said method comprises:
fractionating a sample into a set of fractions using a first liquid phase chromatography device;
fractionating said set of fractions into a set of sub-fractions using a second liquid phase chromatography device;
depositing said set of sub-fractions upon a substrate to form an array of sub-fractions;
interrogating said array with a post-translational modification indicator to identify post-translationally modified sub-fractions; and
assessing any post-translationally modified sub-fractions by mass spectrometry.

11. The method of claim 7, wherein said assessing determines a mass of a post-translationally modified polypeptide.

12. The method of claim 11, wherein said mass identifies said post-translationally modified polypeptide.

13. The method of claim 10, wherein said first or said second liquid phase chromatography device is an ion exchange chromatography device.

14. The method of claim 10, wherein said first or second device is reverse phase chromatography device.

15. The method of claim 7, wherein said post-translational modification indicator binds phosphoproteins.

16. The method of claim 15, further comprising contacting said array with a phosphatase or kinase to verify the presence of a phosphoprotein.

17. The method of claim 7, wherein said post-translational modification indicator is a dye.

18. The method of claim 7, wherein said post-translational modification indicator is a labeled antibody.

19. The method of claim 7, wherein said post-translational modification indicator binds glycoproteins.

20. The method of claim 19, wherein said post-translational modification indicator is a dye.

21. The method of claim 19, wherein said post-translational modification indicator is a labeled antibody.

22. The method of claim 7, wherein said post-translationally modified sub-fractions are subjected to proteolysis prior to said assessing step.

23. The method of claim 7, wherein mass spectrometry employs a time of flight (TOF) spectrometer, Fourier transform ion cyclotron resonance (FTICR) spectrometer, ion trap, quadrupole or double focusing magnetic electric sector mass analyzer, or any hybrid thereof.

24. A system for sample analysis, comprising
a multi-dimensional sample fractionation system for producing sub-fractions of a sample;
a first system for assessing binding of said sub-fractions to a binding agent;
a second system for assessing analyte mass.

25. The method of claim 24, wherein said first system includes:
a device for depositing material on an substrate to form an array;
a post-translational modification indicator;
an array reader.

26. The method of claim 24, wherein said second system includes:
a mass spectrometer.

27. The system of claim 24, wherein said multi-dimensional sample fractionation system includes at least one of ion exchange chromatography device and a reverse phase chromatography device.

28. The system of claim 26, wherein said mass spectrometer system employs a time of flight (TOF) spectrometer, Fourier transform ion cyclotron resonance (FTICR) spectrometer, ion trap, quadrupole or double focusing magnetic electric sector mass analyzer, or any hybrid thereof.

29. A kit comprising:
a first binding agent for evaluating binding of a first portion of a sub-fractions; and
a first reagent for analyzing the analyte mass.

30. The kit of claim 29, wherein said first binding agent is a post-translational modification indicator.
